(19) United States
(12) Patent Application Publication
(10) Pub. No.: US 2012/0109177 A1
(43) Pub. Date: May 3, 2012

(54) BALLOON CATHETER COMPRISING A RADIALLY ASYMMETRICALLY DILATABLE PORTION

(75) Inventor: Jens Ulmer, Zuerich (CH)

(73) Assignee: BIOTRONIK AG, Buelach (CH)

(21) Appl. No.: 13/277,737

(22) Filed: Oct. 20, 2011

Related U.S. Application Data
(60) Provisional application No. 61/407,921, filed on Oct. 29, 2010.

(51) Int. Cl. A61M 29/02

(52) U.S. Cl. 606/192

(57) ABSTRACT

A balloon catheter comprising a dilatable balloon, characterized in that the dilatable balloon comprises at least one radially asymmetrically dilatable portion, wherein the cross-section of the radially asymmetrically dilatable portion has at least one first region and one second region located opposite the first region, and the first and second regions differ from one another in terms of their radial strength of the balloon wall.

### CROSS REFERENCE TO RELATED APPLICATIONS

This invention claims benefit of priority to U.S. patent application Ser. No. 61/407,921, filed Oct. 29, 2010; the contents of which are herein incorporated by reference in their entirety.

### TECHNICAL FIELD

The invention relates to a balloon catheter.

### BACKGROUND

Angioplasty, including percutaneous transluminal angioplasty (PTA) or percutaneous transluminal coronary angioplasty (PTCA), is a method for widening or reopening constricted or blocked blood vessels (usually arteries, but more rarely also veins). Balloon dilation is a common method of angioplasty.

In interventional radiology, cardiology and angiology, balloon dilation in the context of angioplasty is understood to mean a method for widening abnormally constricted blood vessels using a balloon catheter, an angiocatheter with a balloon attached thereto which slowly deploys under high pressure (6-20 bar) only once it reaches the constricted site. Constrictions resulting inter alia from atherosclerotic changes (calcification of the vessels) are thus expanded so that they hinder the blood flow to a lesser degree, if at all.

In this process, the balloon catheters are almost always placed into the stenosis (constriction) from the groin via a guide wire and a guide catheter and are inflated using pressure. This usually eliminates the constriction, and an operation is avoided.

Modern methods in the field of plastics processing make it possible to design and further develop such balloons so as to adapt the quality individually to the needs of patients. The important aspects here are the flexibility of the balloons and their ability to withstand pressure.

The balloon catheters of the prior art are characterized in that they have an axisymmetric radial force distribution during the dilation of the balloon. As a result, the radial force exerted on the vessel walls is substantially evenly distributed along the longitudinal axis of the balloon. However, most stenoses to be treated do not exhibit a radially symmetrical size within the vessel (so-called eccentric stenosis). As a result, when such eccentric stenoses are treated using commercially available balloon catheters, surrounding healthy tissue is unnecessarily subjected to stress and injuries may occur to the vessel wall during the treatment. Such traumatological changes to the vessel wall, including and specifically in healthy tissue, are nowadays considered to be a main trigger for the formation of new tissue (neointimal hyperplasia) and thus to be associated with the risk of restenosis.

### SUMMARY

The object of the present invention is to alleviate or avoid one or more disadvantages of the prior art. In particular, one object of the present invention is to provide means which allow the treatment of stenoses but which subject surrounding healthy tissue to less stress.

The present invention achieves this object by providing a balloon catheter comprising a dilatable balloon, characterized in that the dilatable balloon comprises at least one radially asymmetrically dilatable portion, wherein the cross-section of the radially asymmetrically dilatable portion has at least one first region and one second region located opposite the first region, and the first and second regions differ from one another in terms of their radial strength of the balloon wall (compliance). Preferably, a lower radial strength is achieved by choosing a smaller wall thickness.

The balloon catheter according to the invention is characterized in that the pressure effectively exerted on the vessel wall is distributed asymmetrically during the dilation. During the balloon dilation, regions with a smaller wall thickness produce higher shear forces than regions with a greater wall thickness. As a result, the resulting pressure effectively transmitted to the vessel wall is greater in regions with a smaller wall thickness than in regions with a greater wall thickness relative thereto. The shear force effectively transmitted to the vessel wall will also be referred to as the resulting pressure.

The balloon catheter according to the invention makes it possible to treat constrictions of vessels in a particularly advantageous manner, a relatively high resulting pressure preferably being exerted on the diseased regions of the vessel wall, which are characterized by a high strength, while the resulting pressure exerted on the surrounding healthy parts of the vessel wall is lower due to the increased strength of the balloon wall in this portion and thus due to lower shear forces. A stenosis can thus effectively be eliminated while the surrounding healthy vascular tissue is subjected only to little stress. By using the balloon catheter according to the invention, the formation of new tissue (neointimal hyperplasia) is reduced without the use of medicaments or active substances.

In principle, any known balloon catheter system comprising a dilatable balloon may be used for the balloon catheter according to the invention. In particular, the invention relates to a balloon catheter comprising an inner shaft, to which there is attached at the distal end a dilatable balloon which bears at least partially against an outer surface of the inner shaft in a non-expanded, deflated state. The invention relates in particular to those catheters which have a dilatable balloon, the outer surface of which is pressed at least in parts against a vessel wall after the catheter has been introduced into a vessel and then the balloon has been dilated.

Balloon catheters of the intended type usually have, in addition to an inner shaft and the dilatable balloon, also an outer shaft which extends at least as far as a proximal end of the balloon and is connected to the latter in a fluid-tight manner. Usually provided between the inner shaft and the outer shaft of the catheter is a fluid line which extends in the longitudinal direction of the catheter from its proximal end into the interior of the balloon, and which is obtained for example as a result of the fact that the outer shaft has an inner diameter that is larger than an outer diameter of the inner shaft.

A hollow space which is enclosed by the inner shaft and extends in the longitudinal direction of the inner shaft is provided as a lumen inside the inner shaft. This lumen serves for example to receive a mandrin or a guide wire. The catheter and the guide wire are then configured for example in such a way that the guide wire can emerge from the distal tip of the catheter and can be controlled from the proximal end. The guide wire is deflected for example with the aid of control means so that it can easily be introduced even into blood vessels that branch off. The balloon catheter can then be advanced along the guide wire.

Regardless of the type of catheter, in particular in terms of the configuration of the guide means, balloon catheters have the abovementioned dilatable balloon at their distal end. During the insertion of the balloon catheter, the balloon is compressed and bears tightly against the inner shaft of the catheter. The balloon can be expanded by inflating it with a fluid. This expansion or dilation of the balloon takes place as soon as the balloon has been guided to the intended position. As a result of the expansion of the balloon, a surface of the balloon bears against a vessel wall. This takes place for example for the purpose of widening constrictions of vessels (stenoses) by means of the balloon catheter.

The balloon catheter according to the invention may have an active substance-releasing coating and/or cavity filling at least on parts of the outer surface of the dilatable balloon, preferably only on parts of or on the entire outer surface of the radially asymmetrically dilatable portion(s) of the balloon catheter. Methods for coating balloon catheters and for applying cavity fillings to balloon catheters are known to the person skilled in the art.

The dilatable balloon of the balloon catheter according to the invention has at least one asymmetrically dilatable portion. A portion is understood here to mean a part of the dilatable balloon that radially surrounds the longitudinal axis, extending in the direction of the longitudinal axis. A portion may be delimited for example by two parallel sectional planes (cross-sections) running at an angle of 90° to the longitudinal axis of the dilatable balloon, which are offset by a certain distance in the direction of the longitudinal axis of the balloon. A cross-section through a portion is thus at the same time also a cross-section through the dilatable balloon of the balloon catheter.

The radially asymmetrically dilatable portion of the balloon catheter according to the invention has a cross-section which comprises at least one first region and one second region located opposite the first region. The two regions are located opposite one another when the center points of the regions are located opposite one another on the circumference of the cross-section point-symmetrically through the center point of the cross-section. The first and second region form parts of the circumference of the cross-section through the portion. The cross-section through the portion is preferably such that the cross-sectional circumference includes the maximum radial dimension of the second region. In the simplest case, the entire circumference of the cross-section of the portion is formed of one first and one second region. However, the circumference of the cross-section of the portion may also contain further, different regions. It is also possible that the cross-section of a radially asymmetrically dilatable portion contains more than one first and second region. One second region preferably forms at least 0.01% and no more than 50% of the cross-sectional circumference.

The first and second regions of the cross-section of the radially asymmetrically dilatable portion differ at least in terms of the wall thickness of the respective region. Preferably, the wall thickness of the second region is smaller than the wall thickness of the first region. This difference in the wall thickness means that, during the dilation, the second region transmits a higher resulting pressure to a surrounding vessel wall than the first region of the balloon catheter. Preferably, at the point on the cross-sectional circumference located opposite the minimum wall thickness of the second region, the wall thickness of the first region is greater than the wall thickness at the point of the second region with the minimum wall thickness. This is intended to ensure that the force distribution, brought about by the different shear forces, during the dilation is adapted to the differences in strength of the surrounding vessel wall in such a way that mainly the diseased vessel regions are expanded and the healthy vessel regions are subjected only to little stress. To this end, it is advantageous to select the balloon wall thickness of the second region so that the resulting pressure in the second region is at least equal to, but in the best case lower than, the resulting pressure in the first region. A higher force thus acts on the diseased vessel portions and thus prevents an excessive expansion of the healthy vessel portions.

The balloon catheter according to the invention may be characterized in particular in that the ratio δ of the minimum wall thickness of the second region to the wall thickness of the point on the first region located opposite the minimum wall thickness of the second region is 1>δ≧0.01, preferably 0.95≧δ≧0.1, particularly preferably 0.9≧δ≧0.7.

Here, δ=p/q; where
p=wall thickness at the point with the minimum wall thickness of the second region on the cross-sectional circumference; and
q=wall thickness of the first region at the point located opposite the minimum wall thickness of the second region on the cross-sectional circumference.

The ratio δ is in this case 1>δ≧0.01, preferably 0.95≧δ≧0.1.

This principle works in particular when the balloon is intended to open the stenosis in the to almost fully dilated state. In other words, at the start of inflation all of the tissue is initially elastically expanded. The different expandability of the balloon walls of varying thickness comes into effect only above a certain pressure and when the balloon catheter is almost fully expanded, so as to deform the surrounding tissue also plastically and continuously by the shear forces that occur. In the case of balloon catheters according to the prior art, this plastic deformation takes place only in tissue portions having the lowest strength. Since these are usually the healthy regions, this is intended to be avoided by the present invention in that the healthy vessel portions are protected by an increased strength of the balloon wall.

The balloon catheter according to the invention may also be advantageous in the treatment of so-called vulnerable plaques. These are diseased vessel regions in which the vessel wall contains an unstable encapsulation that tends to rupture. Vulnerable plaques are characterized by an increased risk of thrombosis after plaque rupture. It is therefore desirable to treat these diseased regions with particular care, which is possible using the balloon catheter described here.

The shape, positioning and number of radially asymmetrically dilatable portions of the balloon catheter according to the invention or of the second regions of these portions depends substantially on the shape, position and size of the area to be treated in the vessel.

The balloon catheter according to the invention may have for example just one single radially asymmetrically dilatable portion.

The balloon catheter according to the invention may also have a number of separate, successive or overlapping radially asymmetrically dilatable portions.

It is also possible that one or more of the radially asymmetrically dilatable portions of the balloon catheter according to the invention have more than one second region.

The radially asymmetrically dilatable portions may have one or more second regions which are formed in a certain geometric shape. The second regions may for example be, independently of one another, circular, elliptical or strip-shaped.

The balloon catheter according to the invention may be characterized for example in that it has a single radially asymmetrically dilatable portion and this extends substantially over the entire dilatable balloon.

If the balloon catheter according to the invention has more than one radially asymmetrically dilatable portion, this plurality may be configured and arranged in such a way that the sum of the longitudinal sizes of the individual radially asymmetrically dilatable portions accounts for no more than 50% of the longitudinal size of the dilatable region of the balloon catheter.

Given a suitable configuration and positioning of the second regions, the presence of a plurality of preferentially dilatable second regions on a balloon catheter according to the invention, be it in the context of a single radially asymmetrically dilatable portion or a plurality of radially asymmetrically dilatable portions, may for example also serve to prevent an undesired change in position of the balloon during the dilation. In particular, the regions present in addition to the second region used for therapeutic purposes may be oriented and configured in such a way that an undesired rotational movement of the balloon during the dilation in the vessel is avoided or is at least made more difficult. Such a change in position may occur for example when the preferentially expanded second region meets the lesion to be expanded and is deflected by the latter or slips off the latter before the rest of the balloon has sufficiently expanded to ensure a stable position in the vessel and thus to prevent any undesired rotational movement in the vessel.

In order to make particularly effective use of the advantages of the balloon catheter according to the invention, it is useful to ensure that the balloon catheter, prior to dilation, is radially oriented at the desired location in the vessel in such a way that the preferentially dilatable second region of the radially asymmetrically dilatable portion comes to lie on the site that is to be treated, and not on surrounding healthy tissue.

For the success of the asymmetric force distribution by the balloon catheter according to the invention, it is therefore desirable that the balloon catheter can be precisely positioned within the vessel. Not only does the longitudinal position have to be ensured, but also the balloon must be brought into the correct radial orientation by rotation. It is known to the person skilled in the art that, besides the method described below, further methods and imaging processes exist for achieving the precise positioning of a balloon catheter within the vessel. For example, using intravascular imaging processes (ultrasound, optical coherence tomography) in combination with a balloon catheter, the latter can be precisely oriented within the vessel. Methods also exist for determining the spatial position of a catheter within a lumen with the aid of permanent magnets in the catheter tip.

For a simpler method, the following principle can also be applied:

The surface of the inner shaft and/or outer shaft may have one or more markers which are arranged and oriented in such a way that, during the application of the balloon catheter in the vessel of the individual to be treated, the spatial orientation of the radially asymmetrically dilatable portion and thus in particular the position of the preferentially dilatable second region can be determined. Preferably, the markers are positioned and grouped in such a way that the rotational position of the radially asymmetrically dilatable portion in the vessel can be determined.

In this case, use is preferably made of markers which can be detected and displayed from outside the body by means of imaging processes. Suitable markers are known to the person skilled in the art. These may be for example known X-ray and/or fluorescent markers. In order to allow a reliable detection and display of the three-dimensional position of the balloon catheter in the vessel, preferably more than one marker substance or more than one marker is used, particularly preferably at least two different markers, very particularly preferably at least three different markers. By using a number of different markers, the risk of artifacts in the imaging is reduced.

The markers are preferably arranged in different geometric shapes on the surface of the inner shaft and/or outer shaft of the catheter. The geometric shapes are preferably rings, strips, triangles and/or arrows, the first-mentioned serving as reference systems for the subsequent image processing. These geometric shapes may be configured in such a way that, despite a two-dimensional imaging (in a type of “plan view”), the orientation of the balloon catheter in three-dimensional space can be determined. To this end, different to shapes may be combined with one another. The use of triangles, which may extend in particular around half the circumference of the catheter, is particularly advantageous. In one preferred embodiment, at least three identical or different geometric shapes are applied to and oriented on the surface of the dilatable balloon in such a way that the rotational position of the radially asymmetrically dilatable portion(s) can be determined.

Preference is given to using at least two triangles, the tips of which are arranged in opposite directions. With particular preference, use is made of three triangles, the tips of which are alternately arranged in opposite directions to one another and the base of which is in each case arranged relative to one another in such a way that a clear two-dimensional image that is specific to each rotational position of the balloon catheter is obtained. This image can be analyzed automatically using image analysis processes and can be converted directly into a rotational position. For this, the radio markers at the ends of the balloon are used as reference systems which make it possible to determine clearly both the surface area and the geometric shape (in the two-dimensional display thereof) of the triangular position markings.

### DESCRIPTION OF THE DRAWINGS

### Figures

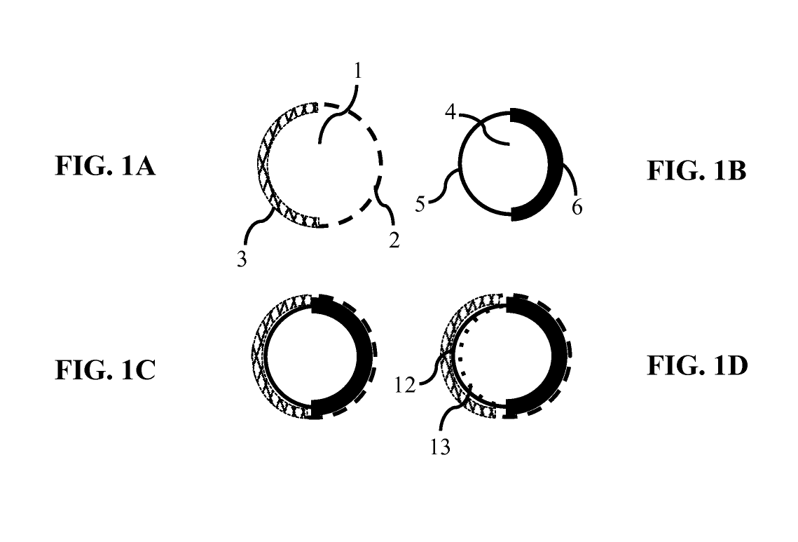

FIGS. 1A-D show a schematic diagram of a first embodiment of the balloon catheter according to the invention and of the use thereof. 1A shows a schematic diagram of a cross-section through a vessel to be treated; 1B shows a schematic diagram of a cross-section through a radially asymmetrically dilatable portion of the balloon catheter; 1C shows the positioning of the balloon catheter according to the invention in the vessel to be treated; and 1D shows the state after the balloon has been positioned and dilated.

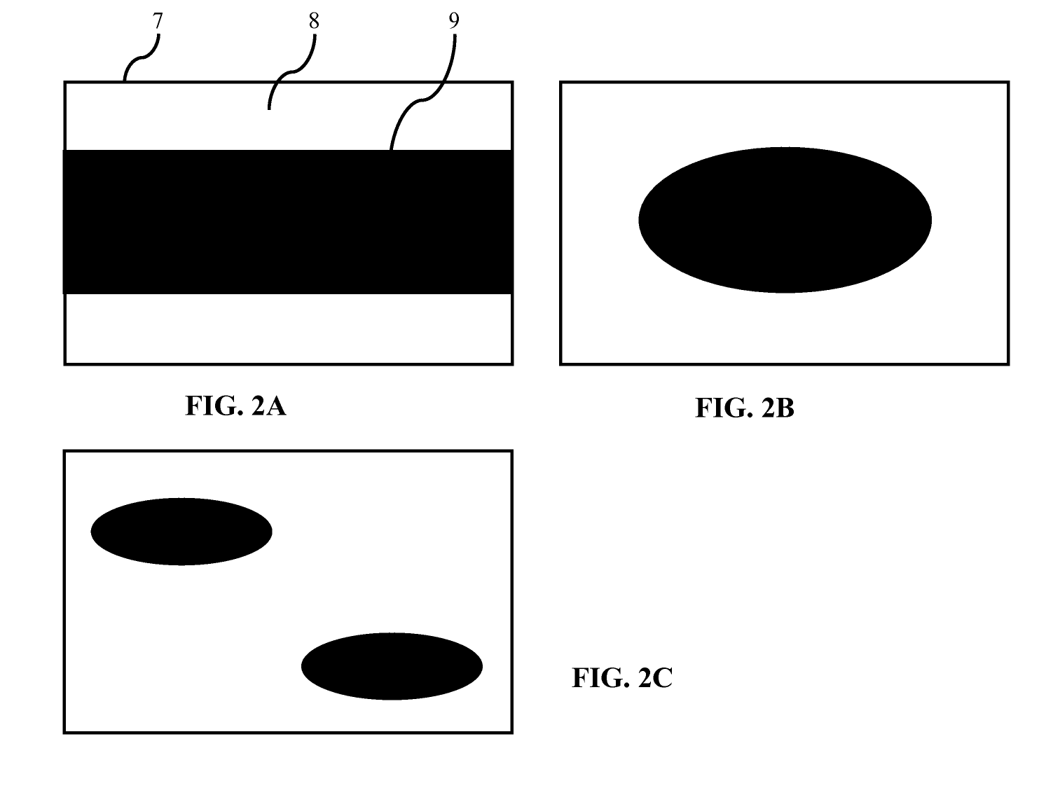

FIGS. 2A-C show a schematic diagram of the configuration of radially asymmetrically dilatable portions of a balloon catheter according to the invention. 2A shows a diagram of a radially asymmetrically dilatable portion with a strip-shaped second region; 2B shows a diagram of a radially asymmetrically dilatable portion with an elliptical second region; and 2C shows a diagram of a radially asymmetrically dilatable portion with two elliptical second regions.

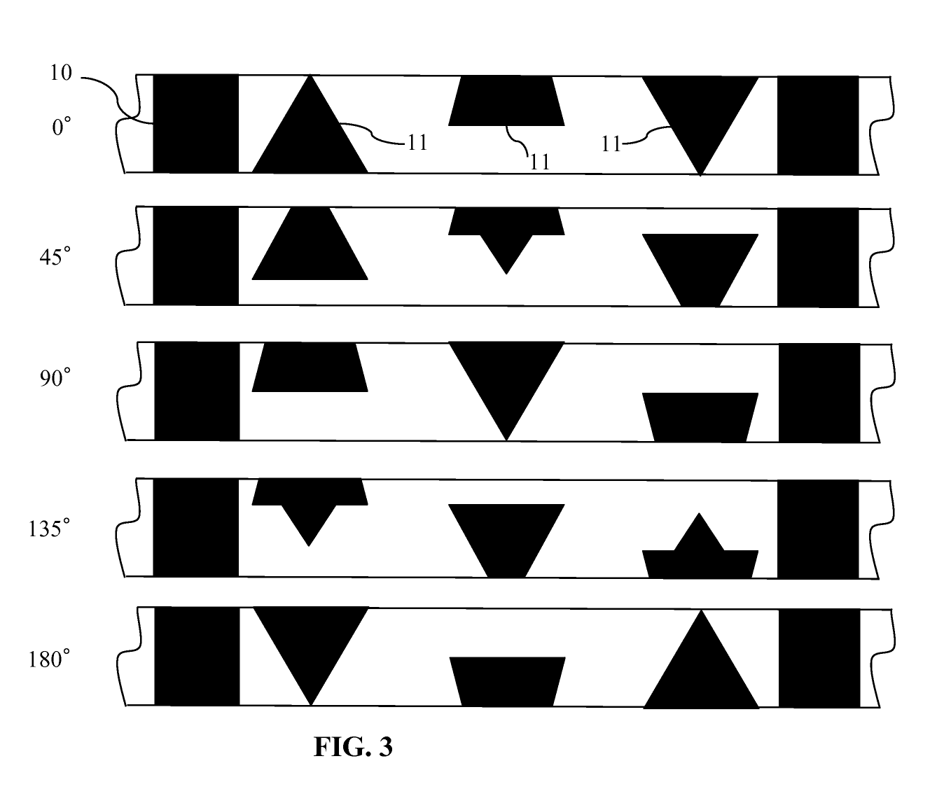

FIG. 3 shows a two-dimensional diagram (as a “plan view”) of a balloon catheter according to the invention with markers arranged on the surface, in different stages of rotation.

### DETAILED DESCRIPTION

The invention will be explained in more detail below with reference to exemplary embodiments.

### Exemplary Embodiment 1

FIG. 1A schematically shows an eccentric stenosis. FIG. 1A shows a cross-section through a vessel with an eccentric stenosis. The vessel lumen 1 is impaired by deposits 3, which nevertheless occur asymmetrically on just one side of the vessel wall. The surrounding healthy regions 2 of the vessel wall do not have any deposits 3 and therefore do not require any treatment.

FIG. 1B shows a first embodiment of the balloon catheter according to the invention, FIG. 1B shows a cross-section through the radially asymmetrically dilatable portion of the balloon catheter. The catheter lumen 4 is shown, which may be provided for the purpose of dilation using a fluid. The catheter lumen 4 is surrounded by a circumferential wall of the dilatable balloon. In the radially asymmetrically dilatable portion, this circumferential wall has in cross-section one first region 6 and one second region 5 located opposite the first to region 6. The first region 6 and the second region 5 differ from one another in terms of the wall thickness and—as a result thereof—in terms of the expandability of the circumferential wall. The wall thickness of the second region 5 is smaller than the wall thickness of the first region 6. As a result, the balloon expands to a greater extent at this point on the circumferential wall than in the first region 6. This means that the resulting pressure of the balloon in the second region 5, which is exerted on the vessel wall, is greater than in the first region 6.

FIG. 1C schematically shows how the balloon catheter according to the invention is positioned in the vessel to be treated. Here, the second region 5 of the balloon catheter points toward the vessel wall area which carries the deposit 3 constricting the vessel lumen 1.

Once the dilatable balloon of the balloon catheter according to the invention has been positioned and dilated, the state shown schematically in FIG. 1D is obtained. The circumferential wall of the dilatable balloon bears against the vessel wall, the resulting pressure exerted on the vessel wall by the circumferential wall in the second region 5 being greater than in the first region 6. Here, 12 indicates the state before the nominal pressure application and 13 describes the position of the final dilated balloon circumference. As a result, therapeutically necessary radial force is exerted on the deposit 3 in a targeted manner, while the surrounding healthy parts of the vessel wall are subjected to less stress and are thus less susceptible to injury.

### Exemplary Embodiment 2

FIG. 2 schematically shows selected configurations of radially asymmetrically dilatable portions of a balloon catheter according to the invention. FIG. 2A shows a radially asymmetrically dilatable portion 7 with one second region 9 in the shape of a strip. Here, the second region 9 extends over the entire expanse along the longitudinal axis of the radially asymmetrically dilatable portion 7. The second region 9 is delimited laterally by a region 8 which has a greater wall thickness than the second region 9.

FIG. 2B schematically shows a radially asymmetrically dilatable portion which has a second region in the shape of an ellipse.

FIG. 2C shows a radially asymmetrically dilatable portion which has two second regions, wherein in this case both second regions are shown in the shape of an ellipse by way of example. The second regions may of course also assume different shapes.

### Exemplary Embodiment 3

FIG. 3 schematically shows an arrangement of markers on the surface of a balloon catheter according to the invention, the markers being positioned and arranged in such a way that the rotational position of the balloon catheter in the vessel can be clearly determined even in a two-dimensional display (as a “plan view”). The balloon catheter has on its surface markers in the form of two strips 10 and at least one triangle 11, preferably two triangles 11, particularly preferably three triangles 11.

Here, the three triangles 11 are oriented and adapted relative to one another in such a way that the rotational position of the balloon catheter in the vessel can be determined in a clear and precise manner from the two-dimensional image thereof. To this end, the three triangles 11 are arranged in an alternating manner in terms of the orientation of the tips. The triangles 11 extend in terms of their size from the tip to the base substantially in each case around half the circumference of the balloon catheter. As shown in FIG. 3, this arrangement of the triangles 11 results in a unique two-dimensional image for each rotational position (0° to 180°), so that the rotational position can be determined on the basis of obtained two-dimensional images. It is sufficient if at least one triangle 11 is imaged. However, in order to minimize the effect of artifact formation during the imaging, it may be useful to use a number of these triangles 11 in a different rotation and orientation relative to one another, it being possible for two triangles to have the same orientation but different rotational positions. Preferably X-ray markers, fluorescent markers and/or combinations thereof are used.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. A balloon catheter comprising a dilatable balloon, characterized in that the dilatable balloon comprises at least one radially asymmetrically dilatable portion, wherein a cross-section of the radially asymmetrically dilatable portion has at least one first region and one second region located opposite the first region, and the first and second regions differ from one another in terms of their radial strength of a balloon wall.

2. The balloon catheter according to claim 1, characterized in that, at a point on the cross-sectional circumference located opposite a minimum wall thickness of the second region, a wall thickness of the first region is greater than the minimum wall thickness of the second region.

3. The balloon catheter according to claim 1, characterized in that the second region comprises no more than 50% and no less than 0.01% of the cross-sectional circumference.

4. The balloon catheter according to claim 1, characterized in that the balloon catheter has more than one radially asymmetrically dilatable portion.

5. The balloon catheter according to claim 1, characterized in that the balloon catheter has a radially asymmetrically dilatable portion with more than one second region.

6. The balloon catheter according to claim 1, characterized in that the radially asymmetrically dilatable portion extends substantially over the entire dilatable balloon.

7. The balloon catheter according to claim 1, characterized in that a sum of longitudinal size of the radially asymmetrically dilatable portions accounts for no more than 50% of a longitudinal size of the dilatable region of the balloon catheter.

8. The balloon catheter according to claim 1, characterized in that the ratio 8 of a minimum wall thickness of the second region to a wall thickness of a point on the first region located opposite the minimum wall thickness of the second region is 1>δ≧0.01, preferably 0.95≧δ≧0.1.

9. The balloon catheter according to claim 1, characterized in that a surface of the dilatable balloon has one or more markers which are applied and oriented in such a way that, during positioning of the balloon catheter in a vessel of an individual to be treated, a spatial orientation of the radially asymmetrically dilatable portion can be to determined, optionally the rotational position of the radially asymmetrically dilatable portion can be determined.

10. The balloon catheter according to claim 9, characterized in that the markers are substances which can be detected and/or displayed by imaging processes and are optionally X-ray and/or fluorescent markers.

11. The balloon catheter according to claim 9, characterized in that at least two different markers are used, optionally at least three different markers.

12. The balloon catheter according to claim 9, characterized in that the markers are arranged in different or identical geometric shapes on the surface.

13. The balloon catheter according to claim 12, characterized in that the geometric shapes comprise rings, strips, triangles and/or arrows.

14. The balloon catheter according to claim 12, characterized in that at least three identical or different geometric shapes are oriented on the surface of the dilatable balloon in such a way that the rotational position of the radially asymmetrically dilatable portion(s) can be determined.
